(19) United States
(12) Patent Application Publication
(10) Pub. No.: US 2026/0335571 A1
(43) Pub. Date: Oct. 29, 2026

(54) SET COMPRISING A DRY POWDER INHALER AND A STORAGE BOX

(71) Applicant: APTAR FRANCE SAS, LE NEUBOURG (FR)

(72) Inventor: Franck POULLAIN, LA HAYE MALHERBE (FR)

(73) Assignee: APTAR FRANCE SAS, LE NEUBOURG (FR)

(21) Appl. No.: 19/103,944

(22) PCT Filed: Aug. 16, 2023
(86) PCT No.: PCT/FR2023/051268

§ 371 Date: Feb. 14, 2025

(30) Foreign Application Priority Data
Aug. 18, 2022 (FR) FR2208358

(51) Int. Cl. A61M 15/00

(52) U.S. Cl. CPC A61M 15/0025; A61M 15/003; A61M 2202/064; A61M 2206/16; A61M 2209/06

(57) ABSTRACT

A set having a dry powder inhaler with a body containing a chamber and a mouthpiece with a dispensing, a reservoir containing a dose of dry powder to be inhaled and having a lateral outlet orifice, the reservoir displaceable between an unloaded position and a loaded position, the diameter of the reservoir being less than the diameter of the chamber, an actuating member for displacing the reservoir from its unloaded position towards its loaded position; and a storage box having a blind hollow body with a bottom wall supporting at least one axial projection which extends axially upwards, the at least one axial projection, in the storage position with the inhaler disposed in the storage box, passing through the mouthpiece and the chamber so as to come into contact with the reservoir in order to lock the reservoir in its unloaded position.

The present invention relates to a set comprising a dry powder inhaler and a storage box.

Dry powder inhalers are well known in the prior art. Various types exist.

A first type of multidose inhaler contains a reservoir receiving multiple doses of powder, the inhaler being provided with dosage means, making it possible, on each actuation, to separate out one dose of this powder from the reservoir in order to bring it into an expulsion duct so that it can be dispensed to the user. Such devices require complicated and therefore expensive dosage means, and dosage accuracy and reproducibility are not guaranteed. In addition, there are risks of contamination of the powder disposed in the reservoir as the inhaler is being used.

Another type of multidose inhaler consists of providing several individual reservoirs each containing one dose of powder, then opening one of those reservoirs each time the inhaler is actuated. £ That implementation ensures that the powder is better sealed, since each dose of the powder is only exposed to the atmosphere at the moment when it is expelled. In order to produce such sets individual reservoirs, different variations have already been proposed, such as blister strips or disks. With regard to opening the individual reservoirs, peeling off or unsticking the closure layer of the blisters has been proposed. That suffers from the disadvantage of difficulty in controlling the forces to be applied in order to guarantee full opening, without running the risk of opening the next reservoir, particularly if the opening means need to be actuated by inhalation. Another solution is to pierce the closure membrane of a blister on each actuation, which requires complicated and therefore expensive piercing means.

In addition, the multidose inhalers described above suffer from the problem of not guaranteeing optimal dispensing effectiveness, with a significant portion of the dose effectively penetrating into the user's lungs in order to have a beneficial therapeutic effect.

In order to make devices less complicated and thus less expensive, single-dose inhalers have been proposed which comprise a single individual reservoir, such as a blister or a capsule, which has to be loaded into the inhaler just before it is used. The advantage of such single-dose devices is that it is not necessary to store all of the doses inside the device, so that the dimensions thereof can be reduced. In contrast, it is more complicated for the user to use, since they are obliged to load a blister or a capsule into the inhaler before each use. Furthermore, other disadvantages specific to these single-dose inhalers exist. Thus, a blister or capsule does not allow large doses, typically greater than 100 mg, to be dispensed. In addition, in particular as regard capsules or blisters to be pierced, opening requires the use of complicated piercing means, and also risks getting residues of pierced membrane in the powder which is dispensed. Furthermore, the emptying performance of the reservoir is not always optimal, since a portion of the powder could remain inside the reservoir during inhalation.

In an attempt to overcome these disadvantages, document WO 98/26828 proposes a single-dose inhaler comprising a cylindrical reservoir provided with a lateral opening, disposed in a cylindrical chamber with a larger diameter, in a manner such that during inhalation, the reservoir is displaced in the chamber in accordance with an Orbital movement, which causes the powder to be expelled. This implementation effectively makes it possible to improve the efficiency of dispensing, with substantially complete emptying of the reservoir and a larger portion of the dose which penetrates effectively into the lungs. Nevertheless, a disadvantage with this device is that in the event of the inhaler being dropped, the reservoir can be displaced in an undesirable manner in the chamber under the effect of the impact, with a risk of losing powder or contamination as the consequence.

The documents WO 2013/008037 A1, WO 2008/001132 A1, TW 2009 24806 A, WO 03/030974 A1 and CN 107469206 A describe other prior art devices.

An objective of the present invention is to provide a set which does not suffer from the aforementioned disadvantages.

In particular, an objective of the present invention is to provide a set of this type which prevents any risk of loss of powder or of contamination in the event of the inhaler being dropped.

Another objective of the present invention is to provide a set of this type which limits or even avoids the risks of contamination and/or pollution of the powder.

Another objective of the present invention is to provide a set of this type which reduces the complexity of use for the user and which guarantees better safety in use.

Another objective of the present invention is to provide a set of this type which is simple and inexpensive to manufacture and to assemble.

The present invention thus provides a set comprising:
a dry powder inhaler comprising a body containing a chamber and a mouthpiece provided with a dispensing orifice through which the user inhales, a reservoir containing a dose of dry powder to be inhaled and comprising a lateral outlet orifice, said reservoir being displaceable between an unloaded position, in which it is at least partially disposed outside said chamber, and a loaded position, in which it is entirely disposed in said chamber, the diameter of said reservoir being less than the diameter of said chamber, an actuating member for displacing said reservoir from its unloaded position towards its loaded position, and
a storage box comprising a blind hollow body comprising a bottom wall supporting at least one axial projection which extends axially upwards, said at least one axial projection, in the storage position with said inhaler disposed in said storage box, passing through said mouthpiece and said chamber so as to come into contact with said reservoir in order to lock said reservoir in its unloaded position.

Advantageously, said blind hollow body is open on the upper side and closed by a lid.

Advantageously, said reservoir is formed by an upper reservoir portion and a lower reservoir portion, which are assembled after filling with the dose of powder.

Advantageously, said lower reservoir portion comprises said lateral outlet orifice.

Advantageously, said at least one axial projection of said storage box cooperates with said upper reservoir portion in the storage position.

Advantageously, in the unloaded position of said reservoir, said lateral outlet orifice of said reservoir is closed by a closure element which is secured to said body of the inhaler.

In accordance with an advantageous variation, said reservoir comprises a closure element which is displaceable and/or deformable with respect to said reservoir between a closed position of said lateral outlet orifice and an open position of said lateral outlet orifice, said closure element being in the closed position when said reservoir is in the unloaded position, and being in the open position when said reservoir is in the loaded position.

Advantageously, said chamber is disposed between an inhalation air inlet and said mouthpiece.

Advantageously, tangential channels bring a flow of inhalation air into said chamber in a tangential manner, which generates an orbital displacement of said reservoir in said chamber.

Advantageously, said reservoir contains a dose of powder greater than 50 mg, in particular greater than 100 mg.

Advantageously, the volume occupied by said reservoir in said chamber in the loaded position is greater than 50% of the volume of said chamber.

Advantageously, the radial diameter of said reservoir is greater than its axial height.

Advantageously, said actuating member is axially displaceable with respect to said body in order to displace said reservoir from its unloaded position towards its loaded position.

Advantageously, said actuating member comprises at least one oblique groove receiving at least one respective lug formed on said body or on an element which is secured to said body, in a manner such that during its axial displacement, said actuating member implements a rotation in said body.

These features and advantages and others of the present invention will appear more clearly during the course of the following detailed description, made with reference to the accompanying drawings, given by way of non-limiting examples, and in which

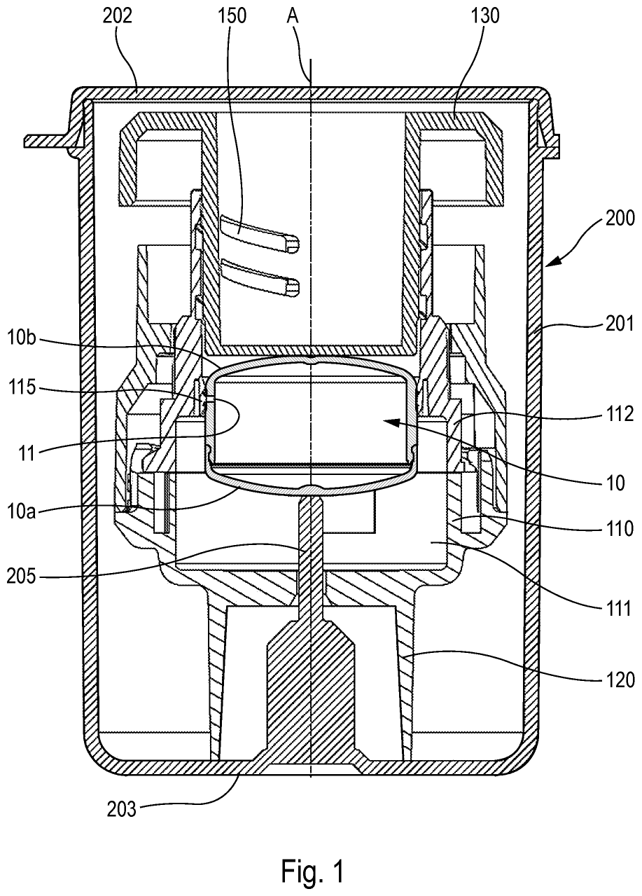

FIG. 1 is a diagrammatic cross-sectional view of a set in accordance with a first advantageous embodiment, in the locked position,

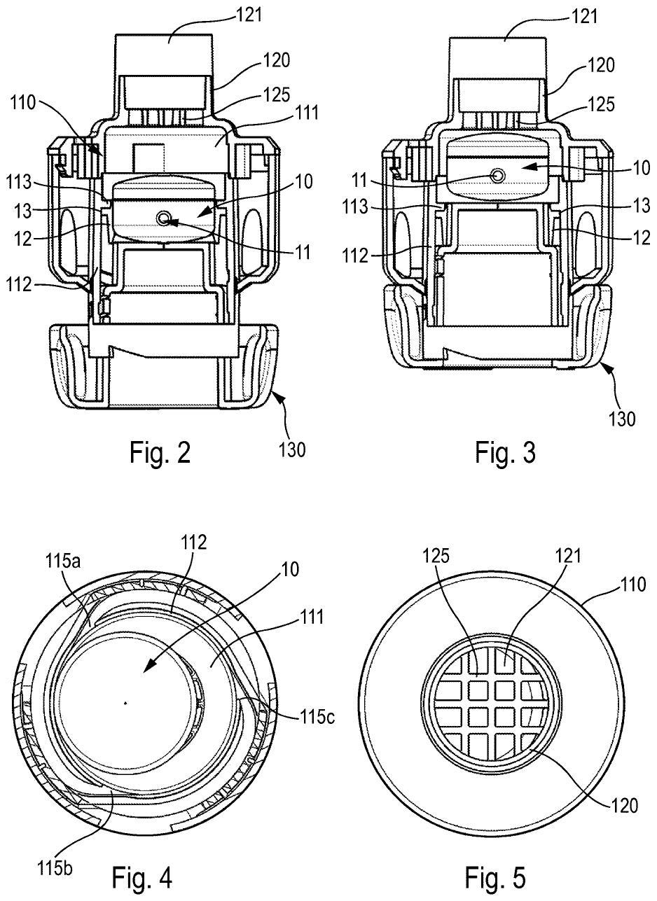

FIG. 2 is a diagrammatic cross-sectional view of an inhaler in accordance with a second advantageous embodiment, in the unloaded position,

FIG. 3 is a view similar to that of FIG. 2, in the loaded position,

FIG. 4 is a diagrammatic view from below of the reservoir in the chamber, in the loaded position, and

FIG. 5 is a diagrammatic view from above of the mouthpiece.

In the description below, the terms “upper”, “lower” and “lateral” are with respect to the upright position of the device shown in FIGS. 1 to 3. The terms “axial” and “radial” are with respect to the longitudinal central axis A of the device, shown in FIG. 1.

The figures describe two embodiments of an inhaler adapted to form part of a set in accordance with the present invention.

FIG. 1 shows a first embodiment of an inhaler.

In this first embodiment, the inhaler comprises a body 110 containing a chamber 111 and a mouthpiece 120 provided with a dispensing orifice 121 through which the user inhales.

Advantageously, the body 110 is formed by two body portions, an upper body portion comprising the mouthpiece 120 and a lower body portion comprising a cylindrical sleeve 112, the chamber 111 being defined by these two body portions fixed to one another.

Preferably, the chamber 111 is disposed between an inhalation air inlet and said mouthpiece 120, in a manner such that when the user inhales through the mouthpiece 120, the air flow will pass through said chamber 111.

The inhaler comprises a reservoir 10 containing a dose of dry powder to be inhaled and comprising a lateral outlet orifice 11. This reservoir 10 is displaceable between an unloaded position, in which it is at least partially disposed outside said chamber 111, and a loaded position, in which it is entirely disposed in said chamber 111. The reservoir 10 comprises a lateral outlet orifice 11 closed by a closure element 115 when the reservoir 10 is not in its loaded position. Advantageously, it is the lower reservoir portion 10b which comprises the lateral outlet orifice 11.

In this first embodiment, the closure element 115 is formed on the cylindrical sleeve 112.

The diameter of the reservoir 10 is less than the diameter of said chamber 111, in a manner such that in the loaded position, the reservoir 10 can be displaced in the chamber 111. Advantageously, tangential channels 115a, 115b, 115c bring said flow of inhalation air into the chamber 111 in a tangential manner, thereby bringing about an orbital displacement of the reservoir 10 in the chamber 111, the reservoir 10 rotating on itself while rotating about the periphery of the chamber 111 along the lateral wall thereof. This movement will allow the powder contained in the reservoir 10 to be expelled, which is entrained towards the mouthpiece and the dispensing orifice by the flow of inhalation air.

Advantageously, the reservoir 10 may contain a dose of powder greater than 50 mg, in particular greater than 100 mg. Optionally, very large doses may be envisaged, in particular greater than 500 mg.

Advantageously, the volume occupied by the reservoir 10 in the chamber 111 in the loaded position is greater than 50% of the volume of the chamber 111.

Advantageously, the radial diameter of the reservoir 10 is greater than its axial height.

Advantageously, the reservoir 10 is formed by an upper reservoir portion 10a and a lower reservoir portion 10b, which are assembled after filling with the dose of powder.

The inhaler also comprises an actuating member 130 for displacing said reservoir 10 from its unloaded position towards its loaded position. This actuating member 130 is axially displaceable with respect to the body 110 in order to push the reservoir 10 from its unloaded position towards its loaded position.

Advantageously, the axial displacement of the actuating member 130 is produced by a rotation of the actuating member 130 in the body 110, for example by means of one or more lugs of the body 110 disposed in one or more oblique grooves of the actuating member 130.

FIGS. 2 and 3 show a second embodiment of the inhaler.

In this second embodiment, the lateral outlet orifice 11 of the reservoir is closed by a specific closure element 12 which is fitted on the reservoir, and which is not part of the inhaler, in contrast to the closure element 115 of FIG. 1.

This closure element 12 protects the powder contained in the reservoir until it is dispensed by actuating the inhaler.

Thus, the closure element 12 is displaceable and/or deformable with respect to the reservoir 10 between closed and open positions of the lateral outlet orifice 11, said closure element 12 being in the closed position when said reservoir 10 is in the unloaded position, and being in the open position when said reservoir 10 is in the loaded position.

Advantageously, in the unloaded position, the closure element 12 abuts against a portion of the body 110 or is secured thereto, so that when the reservoir 10 is displaced towards its loaded position, the closure element 12 remains locked in the body, thereby opening the lateral outlet orifice 11.

In the example of FIGS. 2 and 3, the closure element 12 comprises a radial projection 13 which cooperates with a shoulder 113 of the cylindrical sleeve 112.

Advantageously, the radial projection 13 is disposed to one side of the closure element 12, so that the closure element is not symmetrical. Thus, when the closure element 12 is in the closed position on the reservoir 10, the latter is not symmetrical either; this forms a foolproof key for the insertion of the reservoir 10 into the body 110 of the inhaler, this insertion only being possible in a single orientation of the reservoir 10. This is particularly advantageous when the external shape of the reservoir 10 is substantially symmetrical, but the lateral outlet orifice 11 is not disposed at the axial centre of the reservoir 10, as in the examples in the figures. In fact, in this case, it is desirable to correctly orientate the reservoir 10 in the inhaler for optimal operation, and the asymmetrical shape of the closure element makes it possible to fulfil this function.

Clearly, if the reservoir 10 is symmetrical, with the lateral outlet orifice 11 centred axially, the closure element 12 does not need to be asymmetrical. In contrast, a closure element 12 forming a symmetrical unit with the reservoir 10 would allow the user to insert this unit into the inhaler in both orientations, without any risk of malfunction or less effective powder distribution.

Advantageously, as can be seen in FIG. 5, the mouthpiece 120 may include a grid 125 upstream of the dispensing orifice 121.

In accordance with the invention, a storage box 200 is provided for receiving the inhaler until it is used.

This box 200 comprises a blind hollow body 201 which is open on the upper side and closed by a lid 202.

The blind hollow body comprises, on the lower side, a bottom wall 203 supporting at least one axial projection 205 which extends axially upwards. In the example in FIG. 1, there is a single axial projection 205.

The inhaler is inserted into the transport box 200 with the mouthpiece 120 directed towards the bottom wall, as can be seen in FIG. 1.

In its storage position, with the lid closed, said at least one axial projection 205 passes through the mouthpiece 120 and the chamber 111 so as to come into contact with the reservoir 10, in particular with the upper reservoir portion 10a. This locks the reservoir 10 in the unloaded position, and even in the event of the box 200 being dropped, the reservoir 10 cannot accidentally be displaced into its loaded position.

The set formed by the inhaler and the storage box in accordance with the invention is simple and effective. It is made up of a small number of parts and is thus inexpensive to manufacture and to assemble, and is reliable in use. It allows optimal dispensing of the powder due to the orbital movement of the reservoir during actuation, while guaranteeing the integrity of the powder until it is used.

Various modifications may also be made by a person skilled in the art without departing from the scope of the present invention as defined in the accompanying claims.

1. A set, comprising:
a dry powder inhaler comprising a body containing a chamber and a mouthpiece provided with a dispensing orifice through which the user inhales, a reservoir containing a dose of dry powder to be inhaled and comprising a lateral outlet orifice, said reservoir being displaceable between an unloaded position, in which it is at least partially disposed outside said chamber and a loaded position, in which it is entirely disposed in said chamber, the diameter of said reservoir being less than the diameter of said chamber, an actuating member for displacing said reservoir from its unloaded position towards its loaded position, and
a storage box comprising a blind hollow body comprising a bottom wall supporting at least one axial projection which extends axially upwards, said at least one axial projection, in the storage position with said inhaler disposed in said storage box, passing through said mouthpiece and said chamber so as to come into contact with said reservoir in order to lock said reservoir in its unloaded position.

2. The set as claimed in claim 1, in which said blind hollow body is open on the upper side and closed by a cover.

3. The set as claimed in claim 1, in which said reservoir formed by an upper reservoir portion and a lower reservoir portion, which are assembled after filling with the dose of powder.

4. The set as claimed in claim 3, in which said lower reservoir portion comprises said lateral outlet orifice.

5. The set as claimed in claim 3, in which said at least one axial projection of said storage box cooperates with said upper reservoir portion in the storage position.

6. The set as claimed in claim 1, in which, in the unloaded position of said reservoir, said lateral outlet orifice of said reservoir is closed by a closure element which is secured to said body of the inhaler.

7. The set as claimed in claim 1, in which said reservoir comprises a closure element which is displaceable and/or deformable with respect to said reservoir between a closed position of said lateral outlet orifice and an open position of said lateral outlet orifice said closure element being in the closed position when said reservoir is in the unloaded position, and being in the open position when said reservoir is in the loaded position.

8. The set as claimed in claim 1, in which said chamber is disposed between an inhalation air inlet and said mouthpiece.

9. The set as claimed in claim 1, in which tangential channels bring a flow of inhalation air into said chamber in a tangential manner, which generates an orbital displacement of said reservoir in said chamber

10. The set as claimed in claim 1, in which said reservoir contains a dose of powder greater than 50 mg, in particular greater than 100 mg.

11. The set as claimed in claim 1, in which the volume occupied by said reservoir in said chamber in the loaded position is greater than 50% of the volume of said chamber.

12. The set as claimed in claim 1, in which the radial diameter of said reservoir is greater than its axial height.

13. The set as claimed in claim 1, in which said actuating member is axially displaceable with respect to said body in order to displace said reservoir from its unloaded position towards its loaded position.

14. The set as claimed in claim 13, in which said actuation member comprises at least one oblique groove receiving at least one respective lug formed on said body or on an element which is secured to said body in a manner such that during its axial displacement, said actuation member implements a rotation in said body.
